(19) United States
(12) Patent Application Publication
(10) Pub. No.: US 2025/0018580 A1
(43) Pub. Date: Jan. 16, 2025

(54) AN ISOLATOR MODULE FOR USE IN A SURGICAL ROBOTIC SYSTEM

(71) Applicant: CMR SURGICAL LIMITED, Cambridge, Cambridgeshire (GB)

(72) Inventors: Ivan James Cronin, Cambridge (GB); Benjamin Oliver Brierton, Cambridge (GB)

(21) Appl. No.: 18/711,348

(22) PCT Filed: Nov. 25, 2022
(86) PCT No.: PCT/GB2022/052994

§ 371 Date: May 17, 2024

(30) Foreign Application Priority Data
Nov. 26, 2021 (GB) 2117125.1

(51) Int. Cl. B25J 13/06; A61B 34/37

(52) U.S. Cl. CPC B25J 13/06; A61B 34/37

(57) ABSTRACT

There is provided an isolator module for use in a surgical robotic system which comprises a robot arm and a controller. The isolator module is configured to provide an interface between the robot arm and the controller of the surgical robotic system. The isolator module comprises a first interfacing portion configured to: (i) transfer power to a first cable that is connected to the robot arm, and (ii) transfer data to and from the first cable, and a second interfacing portion configured to: (i) transfer power to a second cable that is connected to the controller, and (ii) transfer data to and from the second cable. The isolator module further comprises a data link connecting the first interfacing portion to the second interfacing portion, the data link being configured to transfer data in both directions between the first interfacing portion and the second interfacing portion whilst providing electrical isolation between the first interfacing portion and the second interfacing portion. The isolator module further comprises an isolated power converter configured to transfer power between the first interfacing portion and the second interfacing portion whilst providing electrical isolation between the first interfacing portion and the second interfacing portion.

### FIELD

This disclosure relates to an isolator module for use in a surgical robotic system, and in particular to an isolator module that is able to transfer power and data between a robot arm and a controller of the surgical robotic system whilst providing electrical isolation between the robot arm and the controller.

### BACKGROUND

A typical surgical robotic system comprises one or more surgical robots connected to a controller that is located within a surgeon's console. Each surgical robot comprises a base unit, a robot arm, and a surgical instrument. The controller provides an interface between the one or more robot arms and a surgeon, thereby permitting the surgeon to control the robot arms. The controller is configured to transfer data signals and power signals to the robot arm. The data signals may comprise control commands transmitted by the surgeon and requests for diagnostics data transmitted from the controller itself. The controller is also configured to receive data signals from the robot arm. The data signals received from the robot arm may comprise a combination of sensor data, position data and diagnostics data.

The controller located at the surgeon's console may be electrically isolated from the one or more robot arms of the surgical robotic system. This electrical isolation reduces the risk of hazardous electrical energy being transmitted from the surgeon's console or other electronic components to the robot arm, and eventually to a patient. In the reverse direction, electrical isolation prevents hazardous electrical energy from the robot arm being transmitted to the surgeon via the surgeon's console.

### SUMMARY

According to a first aspect, there is provided an isolator module for use in a surgical robotic system which comprises a robot arm and a controller, the isolator module being configured to provide an interface between the robot arm and the controller of the surgical robotic system, the isolator module comprising: a first interfacing portion configured to: (i) transfer power from a first cable that is connected to the controller, and (ii) transfer data to and from the first cable; a second interfacing portion configured to: (i) transfer power to a second cable that is connected to the robot arm, and (ii) transfer data to and from the second cable; a data link connecting the first interfacing portion to the second interfacing portion, the data link being configured to transfer data in both directions between the first interfacing portion and the second interfacing portion whilst providing electrical isolation between the first interfacing portion and the second interfacing portion; and an isolated power converter configured to transfer power from the first interfacing portion to the second interfacing portion whilst providing electrical isolation between the first interfacing portion and the second interfacing portion.

The first interfacing portion may comprise a first control unit configured to: transfer power received from the first cable to the isolated power converter; and transfer data between the first cable and the data link.

The second interfacing portion may comprise a second control unit configured to: transfer power received from the isolated power converter to the second cable; and transfer data between the data link and the second cable.

Data may be transmitted through the first and second cables according to a CoaXPress (CXP) data protocol.

Each of the first and second interfacing portions may comprise a CoaXPress (CXP) transceiver module.

The data link may be third cable.

The third cable may be a fibre-optic cable that is configured to transfer data between the first interfacing portion and the second interfacing portion using light.

The light may be infrared light.

The data transferred from the robot arm to the controller may comprise a stream of sensor data.

The first interfacing portion may further comprise a signal conditioning module that is configured to receive a data stream from the data link, extract an embedded clock from that data stream and retransmit a fresh version of the data stream to the first cable using a clean clock.

The first signal conditioning module may be further configured to monitor the quality of the received data stream and transmit data indicating the quality of the received data stream.

The second interfacing portion may further comprise a second signal conditioning module that is configured to receive a data stream from the second cable, extract an embedded clock from that data stream and retransmit a fresh version of the data stream to the data link using a clean clock.

The second signal conditioning module may be further configured to monitor the quality of the received data stream and transmit data indicating the quality of the received data stream.

The isolated power converter may be a DC-DC power converter.

Each of the first and second interfacing portions may comprise a small form-factor pluggable (SFP) interface module.

The module may form part of an isolated ethernet switchboard.

The isolator module may further comprise a microcontroller connected to the first signal conditioning module and configured to receive the results of analysis performed by the first signal conditioning module and to use those analysis results to provide diagnostics information indicating the quality of data being transferred from the robot arm to the controller.

The microcontroller may be further connected to the second signal conditioning module and configured to receive the results of analysis performed by the second signal conditioning module and to use those analysis results to provide the diagnostics information.

The diagnostics information provided by the microcontroller may comprise one or more of the following indications: an overcurrent and undercurrent of each of the first and second interfacing portions; whether or not a first transceiver module and a second transceiver module are present on the isolator module, each of the first and second transceiver modules being configured to transmit and receive data signals to and from the data link; the status of the first and second transceiver modules; whether the first signal conditioning module has been unlocked for use by the isolator module; whether a second signal conditioning module has been unlocked for use by the isolator module; whether there is a fault in the surgical robotic system; the quality of the communication and the signal integrity of the first and second cables; the temperature recorded from first and second temperature sensors located on the first and second interfacing portions, respectively; and the current and voltage that are being provided to the surgical instrument, which thereby provides an indication of quality of the electrical connection to the surgical instrument.

The isolator module may further comprise one or more indicators connected to the microcontroller, the one or more indicators being configured to indicate whether there is a fault in the surgical robotic system.

The one or more indicators may be fault diagnostic light-emitting diodes (LEDs).

The robot arm may comprise a surgical instrument with a camera.

The data transferred from the first interfacing portion to the second interfacing portion via the data link may include control signals and/or interrogations for data from the surgical instrument.

The data transferred from the second interfacing portion to the first interfacing portion via the data link may include one or more of diagnostics data, position data and sensor data.

The sensor data may be image data.

Diagnostics information provided by the microcontroller may comprise one or more of the following indications: an indication of whether the camera has been unplugged from the robot arm; and an indication of the current consumed by the camera.

The module may be located in the surgical robotic system within a unit that comprises the robot arm.

The module may be located in the surgical robotic system within a surgeon's console that comprises the controller.

According to a second aspect, there is provided surgical robotic system comprising: a controller; a robot arm that is controlled by the controller; an isolator module configured to provide an interface between the controller and the robot arm; a first cable connecting the controller to a first interfacing portion of the isolator module, the first cable being configured to: (i) transfer power between the controller and the first interfacing portion, and (ii) transfer data between the controller and the first interfacing portion; a second cable connecting the robot arm to a second interfacing portion of the isolator module, the second cable being configured to: (i) transfer power between the robot arm and the second interfacing portion, and (ii) transfer data between the robot arm and the second interfacing portion; wherein the isolator module is configured to transfer data and power between the first interfacing portion and the second interfacing portion whilst providing electrical isolation between the first interfacing portion and the second interfacing portion.

According to a third aspect, there is provided a surgical robotic system comprising: a controller; a robot arm that is controlled by the controller; the isolator module of as described above; a first cable connecting the controller to the first interfacing portion of the isolator module, the first cable being configured to: (i) transfer power between the controller and the first interfacing portion, and (ii) transfer data between the controller and the first interfacing portion; a second cable connecting the robot arm to the second interfacing portion of the isolator module, the second cable being configured to: (i) transfer power between the robot arm and the second interfacing portion, and (ii) transfer data between the robot arm and the second interfacing portion.

### BRIEF DESCRIPTION ON THE FIGURES

The present invention will now be described by way of example with reference to the accompanying drawings. In the drawings:

FIG. 1 illustrates an arrangement of a surgical robotic system;

FIG. 2 illustrates the electrical isolation of components within the surgical robotic system of FIG. 1;

FIG. 3 illustrates a first exemplary arrangement of an isolator module for use with the surgical robotic system illustrated in FIG. 1;

FIG. 4 illustrates a second exemplary arrangement of an isolator module for use with the surgical robotic system illustrated in FIG. 1;

FIG. 5 illustrates the electrical isolation of components within a surgical robotic system comprising an isolator module as illustrated in FIG. 3 or FIG. 4;

FIG. 6 illustrates two exemplary locations for an isolator module within a surgical robotic system.

### DETAILED DESCRIPTION

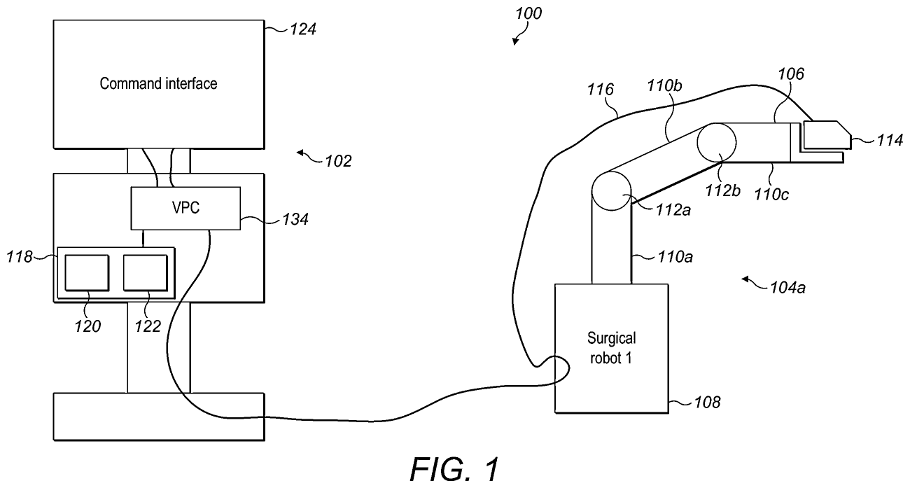

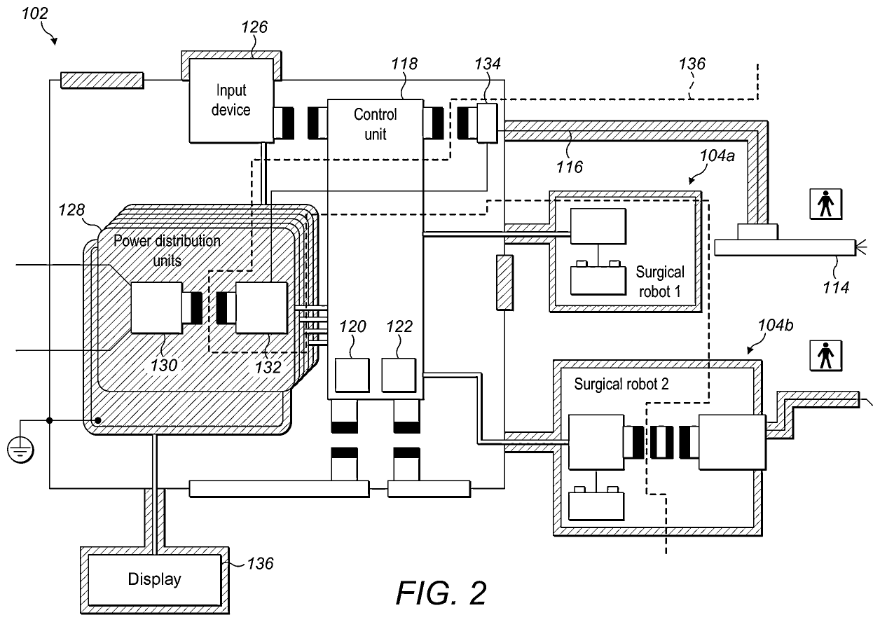

An exemplary surgical robotic system 100 is illustrated in FIGS. 1 and 2. The surgical robotic system 100 comprises a surgeon's console 102 connected to one or more surgical robots 104a, 104b. In FIG. 1, only one surgical robot 104a is connected to surgeon's console 102. In FIG. 2, two surgical robots 104a, 104b are connected to surgeon's console 102. It should be appreciated that the surgical robotic system may comprise one, two or more than two surgical robots connected to a mutual surgeon's console 102.

The surgical robot 104a of FIG. 1 comprises an arm 106 which extends from a base unit 108. The arm has a plurality of rigid limbs 110a-c which are coupled by a plurality of joints 112a, 112b. The plurality of rigid limbs 110a-c may comprise any suitable number of rigid limbs. Similarly, the plurality of joints may comprise any suitable number of joints 112a, 112b. The joints 112a, 112b are configured to apply motion to the limbs 110a-c. Each joint 112a, 112b of the arm 106 has one or more drive sources which can be operated to cause rotational motion at the respective joint. Each drive source is connected to its respective joint by a drivetrain which transfers power from the drive source to the joint.

The most distal limb 110c of the robot arm carries an attachment for a surgical instrument 114. The surgical instrument comprises an end effector for performing an operation on a patient. The end effector may take any suitable form. For example, the end effector may be smooth jaws, serrated jaws, a gripper, a pair of shears, a pair of scissors, a needle for suturing, a laser, a knife, a stapler, a cauteriser, a suctioner or an electrosurgical instrument such as a pair of monopolar scissors. The surgical instrument may alternatively be a device comprising a camera, such as an endoscope. The surgical instrument is configured to transmit feedback data to the surgeon's console 102 via one or more first data links. The feedback data may comprise information from the surgical instrument 114, such as an indication of the position of the surgical instrument, diagnostic data and/or measurements from one or more sensors of the surgical instrument. The feedback data may alternatively or additionally comprise information from the robot arm, such as an indication of the pose of the arm and diagnostic data and/or measurements from one or more sensors positioned along the arm. Where the surgical instrument 114 comprises a camera, the feedback data may comprise a stream of image data. The one or more first data links may be comprised within electrical cabling 116 which is connected to the surgeon's console 102 at a first end and the to surgical instrument 114 at a second end.

Configuration controllers for the drive sources and sensors are distributed within the arm 106 of the surgical robot 104a. The configuration controllers are connected via a second data link to a master controller 118. The second data link, in addition to the one or more first data links, may be comprised within electrical cabling 116. The master controller 118 (which will be referred to from now on as “controller 118”) is located within the surgeon's console 102. The controller 118 comprises a processor 120 and a memory 122. The memory 122 stores, in a non-transient way, software that is executable by the processor 120 to control the operation of the drive sources to cause the robot arm 106 to operate. In particular, the software can control the processor 120 to cause the drive sources (for example via distributed controllers) to drive in dependence on inputs from the sensors and from a surgeon command interface 124. The controller 118 is coupled to the drive sources for driving them in accordance with outputs generated by execution of the software. The controller 118 is connected to the sensors for receiving feedback data from those sensors, and to the command interface 124 for receiving control data from that interface.

The command interface 124 comprises one or more input devices 126 whereby a user can request motion of the end effector in a desired way. The input devices, or input controllers, could, for example, be manually operable mechanical input devices such as control handles or joysticks, touch-operable inputs such as touchscreens, or contactless input devices such as optical gesture sensors or voice sensors. The input devices might monitor eye movement to receive an input. The input devices could, for example, be some combination of these types of input devices. Commands input by the input devices can include movement commands, for example to move the instrument in a particular way, such as lateral movement along an x, y or z axis, and/or a rotation. Such commands can include end effector commands, for example to control an end effector coupled to a distal end of the instrument 114 to operate the end effector. Where the end effector is an endoscope, the commands may include commands to turn on or off the endoscope or commands for the endoscope to zoom in or out.

The software stored in the memory 122 is configured to respond to those inputs and cause the joints of the arm and instrument to move accordingly, in compliance with a predetermined control strategy. The control strategy may include safety features which moderate the motion of the arm and instrument in response to command inputs. Thus, in summary, a surgeon at the surgeon console which suitably comprises the command interface 124 can control the instrument 114 to move in such a way as to perform a desired surgical procedure. Thus, the robot arm 106 functions as a master-slave manipulator where the controller 118 acts as a master controller.

The surgeon's console 102 further comprises one or more power distribution units 128 that are configured to distribute power to different parts of the surgical robotic system. For example, a first power distribution unit may supply power to a first surgical robot 104a, a second power distribution unit may supply power to a second surgical robot 104b, and a third power distribution unit may supply power to the command interface 124. A single power distribution unit may provide power to one or more than one part of the surgical robotic system. In one example, the entire surgical robotic system may be powered by a single power distribution unit. The one or more power distribution units are connected to a power supply such as a mains power supply which generates power for surgical robotic system. Each of the one or more power distribution units has a first interfacing unit 130 that is electrically connected to the power supply, such as a mains power supply. Each of the power distribution units further comprises a second interfacing unit 132 that is electrically connected to the one or more parts of the surgical robotic system to which that unit is configured to supply power.

The first interfacing unit 130 is electrically isolated from the second interfacing unit 132. Power may be supplied from a power distribution unit to a robot arm 104a of the system via one or more power links. The one or more power links may be comprised within electrical cabling 116.

Where one or more surgical instruments in the surgical robotic system 100 comprises a camera, the surgeon's console further comprises a video processor card (VPC) 134 that is configured to process image data received from that camera. The video processor card receives a stream of images from a first data link comprised within electrical cabling 116 and processes those images in order to generate data that can be displayed on a display 136 at the command interface 124 of the surgeon's console 102. The display 136 can be used to provide visual feedback to a surgeon operating the one or more surgical robots of the surgical robotic system.

As mentioned above, each surgical instrument 114 of a surgical robot is used to perform an operation on a patient. The surgical instrument is controlled from the surgeon's console, and control signals from the console are fed to the surgical instrument via data links in electrical cabling 116. Feedback data from the surgical instrument, which may include diagnostics, position, or sensor data, is fed back to the surgeon's console via data links electrical cabling 116. Power is also provided to the surgical instrument (and to the robot arm) from one or more power distribution units via a power link, which may also be comprised within electrical cabling 116. In examples described herein, the electrical cabling 116 is configured to transfer data and power on a single cable rather than transferring data and power on different cables.

The one or more first data links for feeding sensor data, such as image data, back to the surgeon's console should be capable of high-speed data transfer. Typical speed requirements for such data transmission are between 1 and 15 GB/s over a distance of 10 to 40 m of electrical cabling. The speed requirements may be between 3 to 15 GB/s over a distance of 10 to 25 m. Whilst data and power connections are required between the surgeon's console 102 and the one or more surgical robots 104a, 104b of the system, it is beneficial that the surgeon's console itself is electrically isolated from each surgical robot (and therefore the surgical instruments of those robots). This minimises the risk of hazardous electrical energy from the surgeon's console, which has been generated by a power supply such as a mains power supply, being transferred to the patient via the surgical instrument.

In the system illustrated in FIGS. 1 and 2, the electrical isolation between the surgeon's console 102 and the surgical robots 104a, 104b may be provided in part by the VPC 134. The VPC 134 may comprise a serial digital interface (SDI) which uses a pair of electrical transformers to electrically isolate the one or more surgical robots 104a, 104b from the controller 118. Data signals are transmitted between the transformers of the SDI using an induction coupling. A limitation with the use of the VPC to provide electrical isolation is that the SDI comprised within the VPC 134 does not electrically isolate each robot arm 104a from all of the components of the surgeon's console 102. This is illustrated in FIG. 2, in which components inside of the dashed line 138 (on the right-hand side of FIG. 2) are electrically connected to each other, even with the electrical isolation provided by the VPC. These components include the end effector of the surgical instrument 114 (which may include the camera of an endoscope), the electrical cabling 116 which extends between the surgical instrument 114 and the surgeon's console 102, the VPC 134 and the power distribution unit 128 for the VPC. These components are either in conductive contact with the patient or have medium to long term contact with the patient. The electrical isolation illustrated in FIG. 2 is provided deep within the surgeon's console, (i.e., within various modules of the console). This type of isolation provides high level of design complexity, as isolated components must be physically separated from each other within the console.

Further disadvantages of the use of a VPC to provide electrical isolation between one or more robot arms and a controller of a surgical robotic system are listed below:
The SDI transformers require high processing speeds and wide bandwidth requirements, and must be able to bridge large creepage and clearance distances whilst providing high dielectric strength;
The transformers are custom designed parts which are a challenge to manufacture repeatably, presenting challenges for the product supply chain;
The SDI transformers are fragile and prone to damage during assembly and servicing;
Radiofrequency (RF) electrosurgery interference may arise across the SDI transformers; and
The SDI interface is not future proof, meaning that it is unable to support a higher bandwidth for data transmission such as the bandwidth required to transmit 4K video.

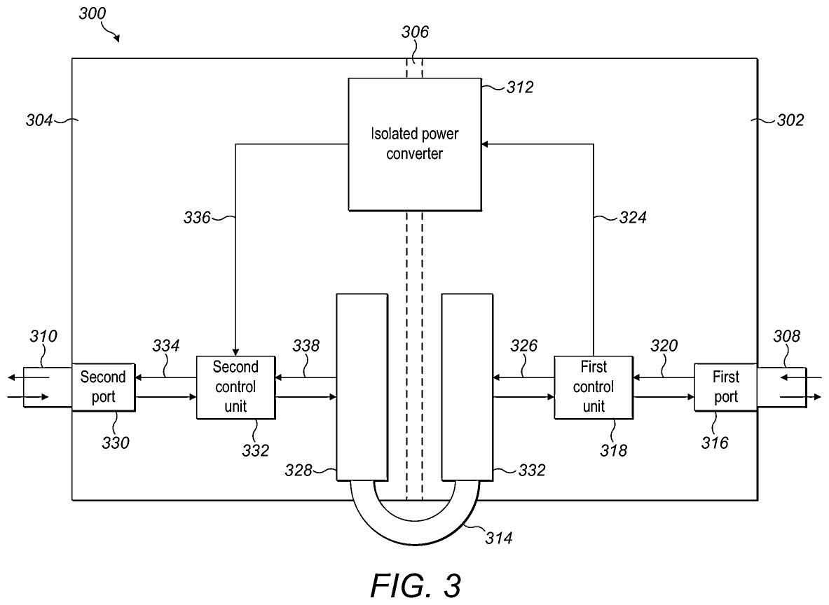

Described below is an improvement to the system described in FIGS. 1 and 2. In order to overcome the limitations of VPC isolation mentioned above, an isolator module has been designed that can be inserted into a surgical robotic system such as the system illustrated in FIGS. 1 and 2. The isolator module is able to transmit power from the controller to the robot arm (or, more specifically, to the surgical instrument of the robot arm) and to transfer data between the controller and the robot arm whilst providing electrical isolation between the controller and the robot arm. The isolator module is configured to provide an interface between a robot arm and the controller of the surgical robotic system. The isolator module may be configured in hardware as any type of suitable module, such as a printed circuit board. An example of such an isolator module 300 is illustrated in FIG. 3.

The isolator module 300 comprises a first interfacing portion 302 and a second interfacing portion 304. The first interfacing portion 302 is electrically isolated from the second interfacing portion 304, as illustrated by the double dashed line 306. The first interfacing portion 302 is located at a first end of the isolator module that is closest to the controller of the surgeon's console. The first interfacing portion 302 is electrically connected to the controller via a first cable 308. The first cable 308 is configured to transfer data in both directions between the controller and the first interfacing portion 302. The first cable 308 is also configured to transfer power in at least one direction from the controller to the first interfacing portion 302. The first cable 308 may be configured to transfer power in both directions between the controller and the first interfacing portion 302. In other words, the first cable 308 is configured to transfer both data and power from the controller to the first interfacing 302 portion, and to transfer at least data from the first interfacing portion to the controller. As it is configured to transfer at least data signals in both directions between the controller and the first interfacing portion 302, the first cable 308 may be referred to as a bidirectional cable. A single cable (first cable 308) is used to transfer both data and power between the isolator module 300 and the controller.

The second interfacing portion 304 is located at a second end of the isolator module that is closest to one of the one or more robot arms of the surgical robotic system. The second interfacing portion 304 is therefore configured to interface with the robot arm via a second cable 310. The second cable 310 is configured to transfer data in both directions between the robot arm and the second interfacing portion 304. The second cable 310 is also configured to transfer power in at least one direction from the second interfacing portion 304 to the robot arm. The second cable 310 may be configured to transfer power in both directions between the robot arm and the second interfacing portion 304. In other words, the second cable 310 is configured to transfer both data and power between the robot arm and the second interfacing portion 304, and to transfer at least data from the robot arm to the second interfacing portion. As it is configured to transfer at least data signals in both directions between the robot arm and the second interfacing portion 304, the second cable 310 may be referred to as a bidirectional cable. A single cable (second cable 310) is used to transfer both data and power between the isolator module 300 and the robot arm.

Each of the first and second cables 308, 310 comprises a data link and a power link. The data link is used to transfer data in both directions along each of the first and second cables. The power link is used to transfer power in at least one direction along each of the first and second cables. By combining the data and power links for the isolator module within a single cable, instead of using a different cable to provide each of these links, the simplicity and compactness of the isolator mechanism can be maximised. Reducing the number of cables that are required is particularly beneficial in a surgical robotic system in which it is important that the robot arm is compact and easily manoeuvrable within an operating theatre. Reducing the number of cables also reduces the trip hazard presented by the surgical robotic system, as well as the capacitive and/or inductive coupling caused by the cables which may harm the patient.

The first interfacing portion 302 is configured to transfer power from the first cable 308 that is connected to the controller to an isolated power converter 312 comprised within the isolator module. The first interfacing portion is further configured to transfer data between the first cable 308 and a data link 314. The second interfacing portion 304 is configured to transfer power from the isolated power converter 312 to the second cable 310 that is connected to the robot arm. The second interfacing portion is further configured to transfer data between that second cable 310 and the data link 314.

The data link 314 is configured to connect the first interfacing portion 302 to the second interfacing portion 304. The data link 314 is configured to transfer data signals in both directions between the first interfacing portion 302 and the second interfacing portion 304. In other words, the data link 314 is configured to transfer data signals from the first interfacing portion to the second interfacing portion, and from the second interfacing portion to the first interfacing portion. At the same time, the data link 314 is configured to provide electrical isolation between the first interfacing portion 302 and the second interfacing portion 304.

The data link 314 may be any suitable link that is able to transfer data signals between the first and second interfacing portions whilst providing electrical isolation between those portions. In one example, the data link may comprise a direct sight laser beam that passes between the first and second interfacing portions. In further example, the data link may be an alternative wireless communication signal, such as a radio frequency (RF) or Bluetooth® signal, that passes between the first and second interfacing portions. In another example, the data link 314 may be a third cable. Where the data link 314 is a third cable, the third cable may bypass the area of electrical isolation 306 between the first interfacing portion 302 and the second interfacing portion 304. That is, the third cable 314 may pass from the first interfacing portion 302 to the second interfacing portion 304 externally of the isolator module. Alternatively, the third cable 314 may be integrated the isolator module and may cross the area of electrical isolation 306 between the first interfacing portion 302 and the second interfacing portion 304. As it is able to transfer data signals in both directions between the first and second interfacing portions, the third cable 314 may be referred to as a bidirectional cable.

The isolator module 300 further comprises an isolated power converter 312. The isolated power converter 312 is configured to transfer power between the first interfacing portion and the second interfacing portion. At the same time, the isolated power convertor 312 is configured to provide electrical isolation between the first interfacing portion and the second interfacing portion. The isolated power converter 312 will also be described in further detail below.

For the purposes of the disclosure in the present application, the term “electrical isolation” refers to an electrical separation or barrier between two circuits, such that currents cannot pass between the first circuit and the second circuit. For example, reference to the first interfacing portion as being electrically isolated from the second interfacing portion of the module demonstrates that the two interfacing portions are electrically separated from each other, such that currents cannot pass from a first one of the two interfacing portions to a second one of those portions.

The first and second interfacing portions of the isolator module will now be described in further detail. The first interfacing portion 302 comprises a first port 316. The first port 316 comprises physical hardware configured to interface with an end of the first cable 308, thereby connecting the first cable 308 to the isolator module 300. The first port 316 therefore provides an electrical connection between the controller of the system and the first interfacing portion 302 via first cable 308. The first interfacing portion 302 further comprises a first control unit 318 that is electrically connected to the first port 316 at an inlet end via a first electrical link 320. The first control unit 318 is electrically connected to the isolated power converter 312 at an outlet end via a second electrical link 324 and is separately electrically connected to a first transceiver module 322 at the outlet end via a third electrical link 326.

The first control unit 318 is configured to receive data and power signals from the first cable 308 via the first port 308. Power signals are received from the controller of the surgical robotic system via the first cable 308. Data signals are also received, in a first direction, from the controller via the first cable 308. The data signals may include control signals and/or interrogations for data from the surgical instrument, such as diagnostic data. The first control unit 318 is configured to separate the power and data signals received from the first cable 308, and to direct them to their respective components on the isolator module 300. For example, the power signals received at the inlet end of the first control unit 318 are forwarded to the isolated power converter 312 via a second electrical link 324. The first control unit 318 is also configured to receive data signals from the robot arm via the second interfacing portion 304. The data signals may include sensor data, diagnostics data that has been requested by the controller.

As data signals are transmitted in both directions across the first interfacing portion 302, the first and third electrical links 320, 326 are configured to transfer data in both directions between the first transceiver module 322 and the first port 316. The first and third electrical links 320, 326 may therefore be referred to as bidirectional electrical links.

The second interfacing portion 304 comprises a second port 330. The second port 330 comprises physical hardware configured to interface with an end of the second cable 310, thereby connecting the second cable 310 to the isolator module 300. The second port 330 therefore provides an electrical connection between the robot arm and the second interfacing portion 304 via the second cable 310. The second interfacing portion 304 further comprises a second control unit 332 that is electrically connected to the second port 330 at an inlet end via a fourth electrical link 334. The second control unit 332 is electrically connected to the isolated power converter 312 at an outlet end via a fifth electrical link 336 and is separately electrically connected to a second transceiver module 328 at the outlet end via a sixth electrical link 338.

The second control unit 332 is configured to receive data and power signals. Power signals are received from the controller of the surgical robotic system via the isolated power converter 312. Data signals are received, in a first direction, from the controller via the first interfacing portion 302. As mentioned above, those data signals may comprise interrogations for data, such as diagnostic data. The second control unit 332 is configured to combine the received data and power signals for transmission to the second cable 310 via the second port 330. The second control unit 332 is also configured to receive data signals from the second cable 310 via the second port 330. The data signals may include position data, diagnostics data and/or sensor data from the robot arm.

As data signals are transmitted in both directions across the second interfacing portion 304, the fourth and sixth electrical links 334, 338, are configured to transfer data in both directions between the second transceiver module 328 and the second port 330. The fourth and sixth electrical links 334, 338 may therefore be referred to as bidirectional electrical links.

Where the data link 314 is a third cable, the third cable may be any type of cable that is capable of transferring data signals between the first and second interfacing portions whilst providing electrical isolation between those portions. In one example, the third cable 314 is a fibre-optic cable. A fibre-optic cable is a cable that comprises one or more strands of fibre that transmit data along the cable as pulses of light. The strands of fibre may be made of any suitable material that is able to transmit light, such as glass or plastic. An advantage of using light to transfer data along the third cable 314 (as opposed to using an electrical signal) is that it provides a robust method of data transfer. That is, the transfer of light along the third cable 314 does not get disrupted due to electromagnetic interferences that may be present in the surgical system. In addition to this, the use of light allows for a fast transmission of data through the isolator module 300, as well as a consistent data transmission across a large range of frequencies. Furthermore, the transceiver modules 322, 328 can be easily replaced depending on the amount of data to be transmitted through the third cable 314. A range of transceivers with a variety of bandwidth capabilities may be available to plug into the module, so that the most appropriate module can be selected for a desired bandwidth. Hence, it is easy to adapt the isolator module to a desired rate of data transfer.

Where the third cable 314 is a fibre-optic cable, the light transmitted through the third cable 314 may be visible light. In other words, the light may be a form of light that can be viewed by the human eye. The light may be near infrared light, which has a frequency range of 300 GHz to 400 THz. Alternatively, the light may be white light, with a frequency of 400 THz to 800 THz. The light may be light of any other suitable frequency. The exact frequency of the light is determined by the frequencies that can be received by the first and second transceivers 322, 328 located at each end of the third cable 314.

The first transceiver module 322 is electrically connected to a first end of the third cable 314 and is configured to transmit data signals from the first control unit 318 through the third cable 314. The first transceiver module 322 is also configured to receive data signals from the third cable 314 from the second interfacing portion 304. In order for data to be transmitted through the third cable 314 whilst electrically isolating the first interfacing portion 302 from the second interfacing portion 304, data must be transmitted through the third cable 314 via a non-electrical signal. Thus, the first transceiver module 322 is further configured to transform electrical signals received from the first control unit 318 into non-electrical signals for transmission through the third cable 314. Similarly, the first transceiver module 322 is configured to transform non-electrical signals received from the third cable 314 into electrical signals to be transmitted to the first control unit 318. Where the third cable 314 is a fibre-optic cable, the first transceiver module 322 is configured to transform electrical signals into optical signals, and vice versa. The first transceiver module 322 may therefore be referred to as an optical transceiver.

The second transceiver module 328 is electrically connected to a second end of the third cable 314 and is configured to transmit data signals from the second control unit 332 through the third cable 314. The second transceiver module 328 is also configured to receive data signals from the third cable 314 from the first interfacing portion 302. Similarly to the first transceiver module, the second transceiver module 328 is configured to transform electrical signals received from the second control unit 332 into non-electrical signals for transmission through the third cable 314. Also, the second transceiver module 328 is configured to transform non-electrical signals received from the third cable 314 into electrical signals to be transmitted to the second control unit 332. Where the third cable 314 is a fibre-optic cable, the second transceiver module 328 is configured to transform electrical signals into optical signals, and vice versa. The second transceiver module 328 may therefore be referred to as an optical transceiver.

In an example, the first and second transceiver modules 322, 328 may be small form-factor pluggable (SFP) transceiver modules. An SFP module is a network interface module comprising a modular slot as an interface that connects its transceiver to the third cable 314. An advantage of using an SFP module as opposed to an alternative transceiver module is that the individual ports of an SFP can be equipped with any suitable type of transceiver. This means that the isolator module can be scaled up easily in the future to provide higher resolution video. In addition to this the use of an SFP fibre link, where the third cable is a fibre-optic cable, is advantageous because it provides fast data transmission speeds. Furthermore, SFP modules are capable of performing equalisation of the data signal received from the third cable. Equalisation is the removal of signal distortion, which improves the quality of the data signal. The SFP modules are also able to advantageously amplify the data signal.

Where the data link 314 does not comprise a cable, the first and second transceiver modules may be any suitable modules that are capable of transferring data between the first and second interfacing portions. For example, the first and second transceiver modules may generate laser, RF, Bluetooth® or any other suitable form of wireless data transmission signals. The first and second transceiver modules may be further configured to receive such suitable signals.

The isolated power converter 312 is configured to transfer power from the first interfacing portion 302 to the second interfacing portion 304 of the isolator module without transferring electricity from the first portion to the second portion. The isolated power converter comprises a first circuit that is electrically connected to the first interfacing portion and a second circuit that is electrically connected to the second interfacing portion. The first circuit and the second circuit are physically isolated from each other. That is, the first circuit is not in physical contact with the second circuit. In other words, the isolated power converter electrically isolates its electrical input (i.e., the surgeon's console) from its output (i.e., the surgical instrument of the robot arm). Whilst the first circuit is electrically and physically separated from the second circuit, the isolated power converter is still able to transfer power from the first circuit to the second circuit. Power may be transferred from the first circuit to the second circuit using any suitable means. In one example, power is transferred through electromagnetic fields using a first transformer located within the first circuit and a second transformer located within the second circuit.

The isolated power converter may be an isolated DC-DC power converter. A DC-DC power converter is an electronic circuit that converts a source of direct current (DC) from one voltage level to another. In other words, where the isolated power converter is an isolated DC-DC power converter, current passing through the first circuit from the controller is direct current, and current passing through the second circuit to the robot arm is also direct current. A direct current power supply is preferred for use in the surgical robotic system because it is more energy efficient than other types of power supply. However, in an alternative example the type of current passing through the converter may be a different type of current, such as alternating current. In this example, the isolated power converter may alternatively be an isolated AC-AC power converter. In further examples, the power converter may be a DC-AC power converter or an AC-DC power converter. In these further examples, either the power source or destination of the power supply could be inverted or rectified as appropriate.

In the example described above, the isolated power converter is configured to transfer power in one direction from the controller to a robot arm of the surgical robotic system. However, in a further example, the isolated power converter may be a bidirectional power converter. In this example, power can be transferred in both directions through the power converter. In other words, in addition to transferring power from the first interfacing portion 302 to the second interfacing portion 304 of the isolator module, the isolated power converter may also be configured to transfer power from the second interfacing portion 304 to the first interfacing portion 302 whilst providing electrical isolation between the first interfacing portion and the second interfacing portion.

The third cable 314 may be a double fibre cable. A double fibre cable is a cable that comprises two fibres: a downlink fibre and an uplink fibre. The downlink fibre is configured to transfer data from the robot arm to the controller. The data from the robot arm may comprise one or more of sensor data, diagnostics data, and position data from the surgical instrument of the robot arm. Where the robot arm comprises a surgical instrument with a camera, the sensor data may be image data from the camera. The image data comprises may comprise a stream of image data (or video data). The camera of the surgical instrument may comprise two channels, a left channel, and a right channel. If this is the case, then the image data transferred from the robot arm to the controller may comprises two separate streams of image data, one for each camera channel.

The uplink fibre is configured to transfer data signals from the controller to the robot arm. The data signals may comprise control signals and/or interrogation signals from the controller. Where the robot arm comprises a surgical instrument with a camera, the control signals may be used to control the performance of the camera. The interrogation signals may also be used to request data indicating the diagnostic status of the camera. The speed of data transfer along the downlink fibre of the third cable 314 may be faster than the speed of data transfer along the uplink fibre. This is because the quantity of data that is transferred from the robot arm to the controller (e.g., image data streams) is greater than the quantity of quantity of data that is transferred from the controller to the robot arm (i.e., control data).

The isolator module is used to provide a data communication link between a robot arm and the controller of that robot arm. More specifically, the data communication link is between the surgical instrument of a robot arm and the controller. The data communication link may conform to a known data protocol. The data protocol may be any suitable protocol that is able to transfer high-frequency sensor data. In one example, the data protocol used by the data communication link is the CoaXpress (CXP) protocol. The CXP protocol is a digital interface standard that is used to communicate high speed image data in machine vision applications. Devices conforming to the CXP protocol are connected together using one or more coaxial cables. Thus, in an example where the data protocol used by the data communication link is the CXP protocol, the first and second cables 308, 310 of the surgical robotic system are coaxial cables. Also in this example, the first and second ports 316, 330 of the isolator module are CXP transceiver modules. That is, each of the first and second ports 316, 330 is configured to both transmit and receive data according to the CXP protocol. The CXP protocol is advantageous for use in the described isolator module as it supports a fast transfer of data. Data transfer speeds offered by coaxial cables are of at least 6.25 Gb/s per cable. A further advantage of using the CXP protocol is that this protocol allows for data to be transferred through the isolator module without having to decode the data in the CXP stream as it passes through the module, and specifically between the first interfacing portion 302 and the second interfacing portion 304. This means that the design of the isolator module can be simplified, as it does not need to include encoding and decoding logic. The CXP protocol is also associated with reduced latency.

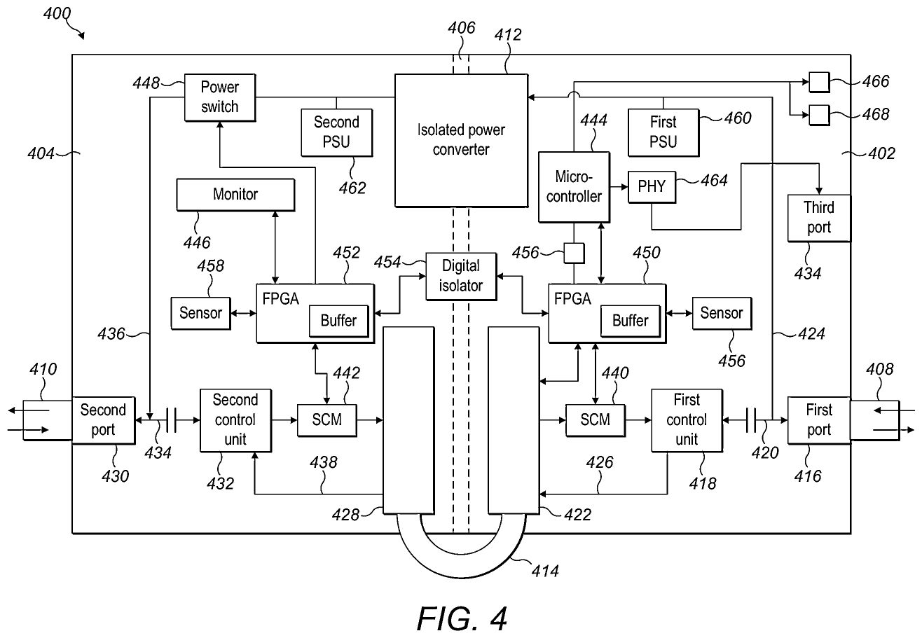

A more detailed example of an isolator module is illustrated in FIG. 4. The isolator module comprises a number of components that are the same as corresponding components in the module illustrated in FIG. 3. These components have been assigned corresponding reference numerals to their counterparts in FIG. 3. For example, first interfacing portion 402 in FIG. 4 corresponds to first interfacing portion 302 in FIG. 3.

As mentioned above, the data signals transmitted between the robot arm and the controller of the surgical robotic system may form a stream of sensor data. More specifically, where the surgical instrument attached to the robot arm comprises a camera, the sensor data sent from the robot arm to the controller may comprise a stream of image data. Where the data is a stream of sensor data, transmission of the stream of data through the cables of the surgical robotic system may introduce “jitter” into the data stream. Jitter is described as the deviation in the periodicity of a received data signal from the original periodicity of that signal. Jitter may be introduced as sensor data passes through the second cable 410 from the robot arm to the second interfacing portion 404 of the isolator module, through the third cable from the second interfacing portion 404 to the first interfacing portion 402 of the module (where the data link is a third cable), and from the first interfacing portion 402 to the first cable 408. In other words, the use of any wired transmission means to transfer data signals introduces jitter into a data signal. Jitter has a negative impact on the quality of a received data stream, as it causes a distortion of the signals comprised within this stream.

To avoid the degradation in the quality of the data stream caused by jitter, the isolator module may comprise one or more signal conditioning modules. For example, in FIG. 4, the first interfacing portion 402 of the isolator module comprises a first signal conditioning module 440. The first signal conditioning module 440 is electrically connected to the first transceiver module 422 at a first end, and to the first control unit 418 at a second end. The first signal conditioning module 440 is configured to receive data from the first transceiver module 422 that has passed through the third cable 414 from the second interfacing portion 404 of the isolator module. In other words, the first signal conditioning module 440 is configured to receive data from the robot arm, or specifically the surgical instrument of the robot arm. The first signal conditioning module 440 is further configured to extract an embedded clock from the data stream received from the first transceiver module 422 and retransmit a fresh version of the data stream to the first control unit 418 (and therefore the first cable) using a clean clock. In doing so, the first signal conditioning module 440 is able to reduce the deviation of the periodicity of the received data stream from its original periodicity, thereby reducing jitter in the data stream. This process is otherwise referred to as “re-timing” of the received signal. In performing its re-timing operation, the first signal conditioning module 440 is further able to remove distortion from the data signal, which improves the overall quality of the data signal.

The second interfacing portion of the isolator module further comprises a second signal conditioning module 442. The second signal conditioning module 442 is electrically connected to the second control unit 432 of the second interfacing portion at a first end, and to the second transceiver module 428 at a second end. The second signal conditioning module 442 is configured to receive data from the second cable 410 via the second port 430. In other words, the second signal conditioning module 442 is configured to receive data from the robot arm, or specifically the surgical instrument of the robot arm. The second signal conditioning module 442 is further configured to extract an embedded clock from the data stream received from the second port 442 and retransmit a fresh version of the data stream to the second transceiver module 428 (and therefore the second cable) using a clean clock. Thus, similarly to the first signal conditioning module 440, the second signal conditioning module 442 is configured to re-time it's received data stream, thereby reducing jitter, and improving the quality of the stream. In performing its re-timing operation, the second signal conditioning module 442 is further able to remove distortion from the data signal, which advantageously enhances the data signal.

In an example, each data signal passing through the isolator module 400 is re-timed a single time, either by the first signal conditioning module 440 or by the second signal conditioning module 442. In an alternative example, each data signal passing through the isolator module is re-timed twice: a first time by the second signal conditioning module 442 after it has passed through the second cable 410 to the isolator module, and a second time by the first signal conditioning module 440 after it has passed through the second and third cables.

In addition to performing the signal conditioning operations described above, the first and second signal conditioning modules 440, 442 may be further configured to monitor the quality of their received data streams for diagnostic data. That is, the signal conditioning modules are configured to detect diagnostic data received from the robot arm. The signal conditioning modules may be further configured to analyse the diagnostic data in order to characterise and analyse the performance of the robot arm. Diagnostic data from the robot arm may be used, for example, to characterise the quality of cabling in the robot arm, or more specifically the cabling in the surgical instrument. The first and second signal conditioning modules 440, 442 may then be configured to transmit data indicating the quality of the stream of data. The data indicating the quality of the stream of data may be used to generate a graphical representation of that quality data. The graphical representation may be generated by the control unit 118 on the surgeon's console, for example. Alternatively, the graphical representation may be generated by the signal conditioning modules 440, 442. In one example, the graphical representation may be an eye diagram. An eye diagram is generated by overlaying different segments of a data signal that are driven by a master clock. Such diagrams can be used to identify jitter, which is illustrated as misalignment of the visual representation of data signal segments on the eye diagram.

The isolator module may further comprise a microcontroller 444. The microcontroller 444 may be connected to at least one of the first signal conditioning module 440 and the second signal conditioning module 442. In FIG. 4, the microcontroller 444 is illustrated as being indirectly connected to the first signal conditioning module 440. In an alternative example, the microcontroller may be connected to both the first signal conditioning module 440 and the second signal conditioning module 442. The microcontroller 444 may be indirectly connected to the first signal conditioning module 440 via a first integrated circuit 450. The microcontroller 444 may be indirectly connected to the second signal conditioning module 442 via a second integrated circuit 452. The first integrated circuit 450 may therefore be located on the first interfacing portion 402 of the isolator module, with the second integrated circuit 452 being located on the second interfacing portion 404.

In addition to being connected to the first signal conditioning module 440 and the microcontroller 444, the first integrated circuit 450 may be connected to a first sensor 456 located on the isolator module. The second integrated circuit 452 may be connected to a second sensor 458 in addition to the second signal conditioning module 442 and the microcontroller 444. The first and second integrated circuits 450, 452 may be configured to read periodic data from the first and second signal conditioning modules, respectively. The first and second integrated circuits 450, 452 may be further configured to read data from the first and second sensors 456, 458, respectively. The first sensor 456 may be configured to provide an indication of the status of the first interfacing portion. The second sensor 458 may be configured to provide an indication of the status of the second interfacing portion. Data from the first and second sensors 456, 458 may be compared, by either the first integrated circuit 450 or the microcontroller 444, to determine whether the isolator module is operating correctly. In one example, the first and second sensors are temperature sensors configured to sense the temperature of the first and second interfacing portions, respectively. In this example, temperature data from the first and second sensors 456, 458 may be compared by either the first integrated circuit 450 or the microcontroller 444. A significant deviation between the temperature data sensed by the first sensor 456 and that sensed by the second sensor 458 may be used to indicate a problem with the isolator module.

The first and second integrated circuits 450, 452 may be further configured to read data from the first and second transceiver modules 422, 428. More specifically, the first and second integrated circuits 450, 452 may be configured to receive data from the first and second transceiver modules 422, 428 indicating the status of those modules, such as their receiving or transmitting capabilities. In an example, as illustrated in FIG. 4, the first and second integrated circuits may be field programmable gate arrays (FPGAs).

The first integrated circuit 450 may be connected to the second integrated circuit 452 across the electrical isolation 406 via a digital isolator 454. The digital isolator 454 provides electrical isolation between the first and second interfacing portions of the isolator module whilst allowing data to pass between the first and second portions of the isolator module. More specifically, the digital isolator allows diagnostic data to pass between the first and second integrated circuits. In FIG. 4 the digital isolator 454 is illustrated as an independent component to the first and second transceiver modules 422, 428. However, in an alternative example the digital isolator may be comprised within the first and second transceiver modules 422, 428.

The microcontroller 444 may be configured to receive the diagnostic data detected by one or more of the signal conditioning modules 440, 442. Where the microcontroller 444 is configured to receive diagnostic data from both signal conditioning modules, the microcontroller may be connected to an interrupter switch 456 which selects an integrated circuit to be interrogated by the microcontroller at a specific point in time. The interrupter switch 456 therefore allows the microcontroller to select the integrated circuit from which it is to receive data. Diagnostic data may be analysed by the one or more signal conditioning modules 440, 442 before it is provided to the microcontroller 444. In other words, the microcontroller 444 may receive data transmitted by the first and second signal conditioning modules 440, 442 indicating the quality of the data received by those modules. Additionally, or alternatively, the diagnostic data may be analysed by an integrated circuit 450, 452 located on the isolator module before it is passed to the microcontroller.

The microcontroller 444 is configured to use the information and analytics received from the first and second integrated circuits 450, 452 to provide diagnostics information indicating the quality of the download link from the robot arm. That is, the microcontroller is configured to receive the results of analysis performed by the first signal conditioning module and the FPGA, and to use those analysis results to provide diagnostics information indicating the quality of data being transferred from the robot arm to the controller. More specifically, the microcontroller 444 is configured to use its received information to provide an indication of the quality of sensor data obtained from the surgical instrument of the robot arm. Where the surgical instrument comprises a camera, the indication may be an indication of the performance of the camera. The indication of the quality of sensor data generated by the microcontroller 444 may comprise one or more of the following indications:
The overcurrent and undercurrent of each of the first and second interfacing portions;
Whether or not the first and second transceiver modules of the interface module are present on the isolator module;
The status of the first and second transceiver modules;
Whether the first signal conditioning module has been unlocked for use by the isolator module. The term “unlocked” refers to a desynchronisation of a data stream by a signal conditioning module;
Whether the second signal conditioning module has been unlocked for use by the isolator module;
Whether there is a fault in the robot arm, such as a fault in the surgical instrument;
Where the surgical instrument comprises a camera, whether the camera has been unplugged from the robot arm;
Where the surgical instrument comprises a camera, an indication of the current consumed by the camera.
The quality of the communication and the signal integrity of the first and second cables;
The temperature recorded from first and second temperature sensors located on the first and second interfacing portions, respectively;
The current and voltage that are being provided to the surgical instrument, which thereby provides an indication of quality of the electrical connection to the surgical instrument.

The overcurrent and/or undercurrent of the first and second interfacing portions may be measured using one or more current-sensing comparator. The current-sensing comparator detects overcurrent by measuring the voltage developed across a shunt resistor and comparing that voltage to the threshold voltage input level. The current-sensing comparator may be comprised within a monitor 446 that monitors the voltage and current consumed by the surgical instrument. As illustrated in FIG. 4, where the surgical instrument comprises a camera, the monitor 446 is configured to monitor the voltage and current consumed by the camera. The monitor 446 is connected to a power switch 448 via the second integrated circuit 452. The power switch 448 may be switched off or on by the second integrated circuit 452 depending on whether power needs to be supplied to the robot arm (and surgical robot) via the second interfacing portion 404.

The microcontroller 444 is further configured to transmit its indication of the quality of sensor data obtained from the surgical instrument of the robot arm off of the isolator module. It may send this indication off of the isolator module as data packets. The microcontroller 444 may send such data packets periodically (i.e., at regular time intervals). It may implement the sending of packets off of the isolator module using a third port 434. The third port 434 may be an ethernet port. The third port 434 may enable data packets from the microcontroller to be sent to an External Interface Controller (EIC) over an Ethernet connection. To do this, the microcontroller may be connected to the third port 434 via a physical layer 464. The physical layer 464 may provide an interface between the third port 434 and the microcontroller 444. The EIC may be located at the surgeon's console. By sending diagnostic data packets from the isolator module to the EIC located at the surgeon's console, information indicating the status of the robot arm may be fed back to the surgeon's console from the isolator module. The information may then be used at the surgeon's console to perform preventative maintenance of the robot arm, or alternatively to provide a notification that a component of the arm should be replaced and/or reconnected. In a further example, the information may be used at the surgeon's console to perform debugging operations.

The isolator module may further comprise one or more indicators configured to indicate whether there is a fault in the surgical robotic system. The one or more indicators may be connected to the microcontroller 444 such that the results of analytics performed by the microcontroller can be communicated using the indicators. In one example, the one or more indicators may comprise microphones configured to provide an audible notification of a fault on the isolator module. In another example, the one or more indicators may comprise a visual indicator configured to provide a visual notification of a fault on the isolator module. In a specific example, the one or more indicators may be fault diagnostic light-emitting diodes (LEDs). In the example illustrated in FIG. 4, the isolator module comprises two indicators 466, 468. In alternative examples, the isolator module may comprise one indicator or more than two indicators.

The exemplary isolator module of FIG. 4 further comprises first and second power supply units (PSUs) 460, 462. Each of the first and second PSUs are configured to convert power from one form into another form (e.g., from AC power into DC power), and to supply that power to components of a respective interfacing portion of the isolator module. The first and second PSUs are configured such that the quantity of power that they supply to respective components of the isolator module is of a predetermined amount. The predetermined amount is such that electrical components of the isolator module are sufficiently powered without being overloaded with power. In other words, the first and second PSUs ensure that power provided to the electrical components of the isolator module is within the acceptable limits for their operation.

Each of the first and second PSUs are configured to receive power from the surgeon's console via the first cable 408, convert that power from a first form (e.g., AC power) into second form (e.g., DC power), and distribute that power amongst the electrical components on each respective interfacing portion of the isolator module. The first PSU may convert and supply power for electrical components of the first interfacing portion 402, and the second PSU may convert and supply power for electrical components of the second interfacing portion 404. In some examples, the first PSU may supply power for electrical components of the first interfacing portion 402 without converting that power from one form into another form. Similarly, the second PSU may supply power for electrical components of the second interfacing portion 404 without converting that power from one form into another form.

The path of power to be provided to electrical components of the isolator module is as follows. Power is transferred from the surgeon's console via the first cable 408 and is received by the isolator module at the first port 416. It then passes along a first electrical path 424 to the first PSU 460. The first PSU 460 is configured to supply a predetermined amount of power for the electrical components of the first interfacing portion 402. Power that is not supplied by the first PSU 460 to the electrical components of the first interfacing portion 402 passes through the isolated power converter 412 and along a second electrical path 436. The second electrical path 436 connects the isolated power converter 412 to the second PSU 462. The second PSU 462 is configured to supply a predetermined amount of power for the electrical components on the second interfacing portion 404. Power that is not supplied by the second PSU 462 to the electrical components of the second interfacing portion 404 continues along the second electrical path 436 and reaches the second port 430, from where it is supplied to the robot arm via the second cable 410.

In one example, as illustrated in FIG. 4, each of the first and second PSUs may be configured to supply power to multiple electrical components of the first and second interfacing portions, respectively. For example, the first PSU 460 may be configured to supply power to all of the electrical components of the first interfacing portion. Similarly, the second PSU 462 may be configured to supply power to all of the electrical components of the second interfacing portion. In an alternative example, each electrical component of the first and second interfacing portions may require a respective PSU. Thus, in this alternative example, each of the first and second interfacing portions may comprise multiple PSUs. The amount of power that is supplied to each electronic component of the isolator module by its respective PSU is determined by the datasheet, or specification, of that component. For example, a component that requires 2.5V to operate may be connected to a 2.5V PSU that is configured to convert and supply 2.5V of power to that component. The voltage suppliable by a PSU may be of any suitable quantity. Exemplary quantities are 1.25V, 2.5V and 3.3V.

Each of the isolator modules described above with reference to FIGS. 3 and 4 is a single module that can provide both electrical isolation over a data link and electrical isolation over a power link. The combination of both of these isolation mechanisms onto a single isolation device improves the overall compactness of the apparatus for transferring data and power between the robot arm and the controller, when compared to the integration of these isolation mechanisms onto separate devices. This is particularly beneficial in surgical robotic systems, which have spatial limitations due to space taken up by the vast quantity of sensors and control devices that these systems require. Additional advantages afforded by the use of an isolator module as described above are an increased speed of data transfer, a reduction in the number of cables that are needed transfer data through the surgical robotic system, the implementation of power and diagnostics for the surgical instrument and the ease of adaptation of the module to fulfil different bandwidth requirements. Furthermore, an isolator module can be placed at various locations within the surgical robotic system, which provides flexibility with respect to the routing of cables within the system.

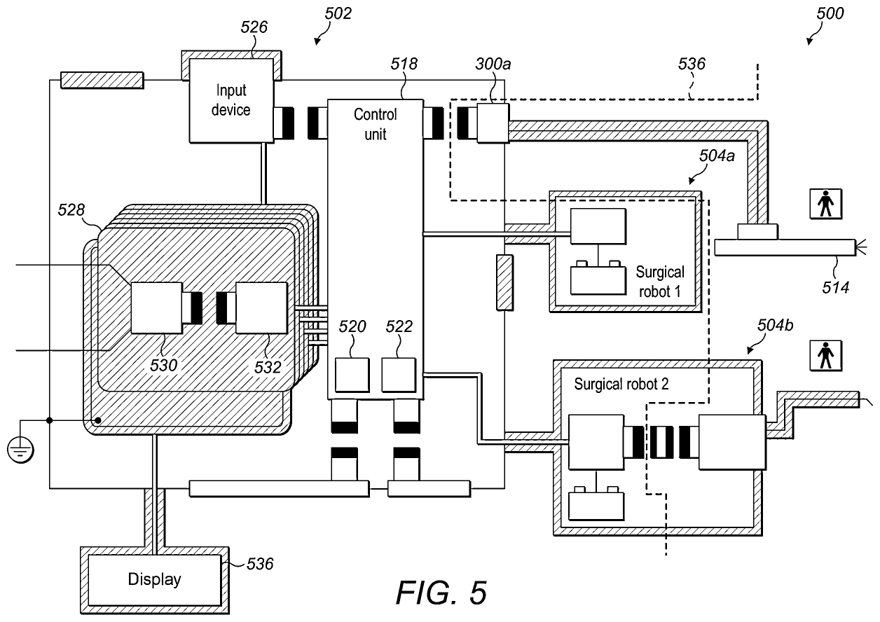

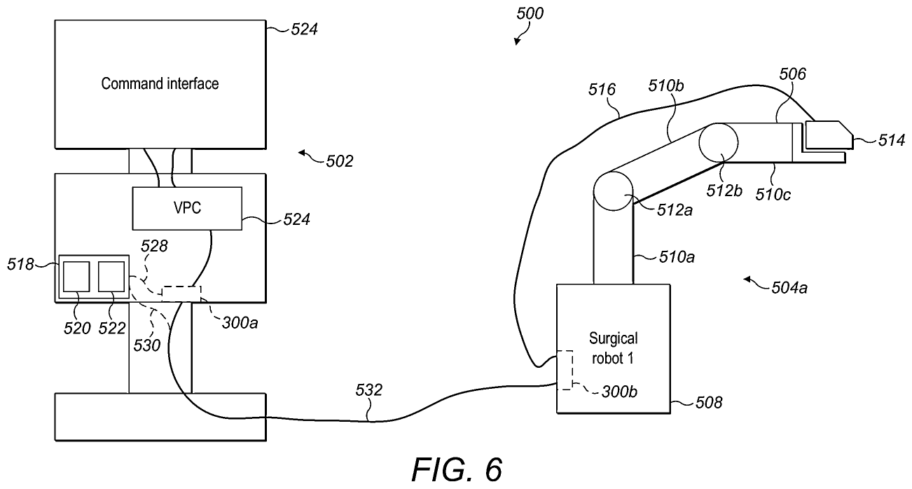

FIGS. 5 and 6 illustrate a surgical robotic system 500 comprising at least one isolator module 300a, 300b as illustrated in FIGS. 3 and 4. Reference numerals 300a, 300b in FIG. 6 denote two alternative placements for the isolator module within the surgical robotic system. FIG. 5 illustrates the electrical isolation of components in a surgical robotic system comprising such an isolator module. The surgical robotic system 500 comprises a number of components that are the same as corresponding components in the system illustrated in FIGS. 1 and 2. These components have been assigned corresponding reference numerals to their counterparts in FIGS. 1 and 2. For example, first surgeon's console 502 in FIGS. 5 and 6 corresponds to surgeon's console 102 in FIGS. 1 and 2.

It can be seen that, as opposed to the system illustrated in FIG. 2, the presence of an isolator module 300a, 300b (or 400a, 400b) provides electrical isolation between a robot arm 504a and all of the components of the surgeon's console 502, apart from the isolator module itself. That is, the isolator module 300a, 300b provides electrical isolation between a surgical instrument 514 of the system and all of the power supply units 528 (and thus the main power supply) of the surgeon's console 502. The isolator module 300a, 300b also provides electrical isolation between a surgical instrument 514 of the system and the controller 518 of the surgeon's console 502. Thus, the isolator module prevents hazardous electrical energy from being transmitted from the surgeon's console 502 to the surgical instrument 514, and eventually to a patient. In the reverse direction, the isolator module 300a prevents electrical energy from being transmitted from the robot arm 506 to the surgeon via the surgeon's console 502. In addition to this, the isolator module 300a, 300b provides enhanced data transmission speeds and a modular design that ensures design flexibility of the isolation mechanism. The isolator module also provides a compact mechanism for compiling and transmitting diagnostic data, as the diagnostic data is compiled on the same component.

In a first example, the isolator module 300a may be located in the surgical robotic system within a surgeon's console 502 that comprises the controller 518. An advantage of placing the isolator module in this first location is that it provides the data signal that is passed through the module with an improved immunity to electrosurgery interference. A further advantage is that the signal conditioning to be performed on the module may be completed shortly before the signal is displayed on a monitor of the console, which improves the quality of that displayed signal. In a second, alternative, example the isolator module 300b may be located within a unit that comprises the robot arm. That is, the isolator module may be located within a surgical robot 504a. In a more specific example, the isolator module may be located within the base unit 508 of the robot arm. An advantage of placing the module in this second location is that it provides better prevention of capacitively coupled electrosurgery energy from reaching the patient.

For each of the two exemplary locations provided above, the surgical robotic system further comprises a first cable 528, 530 connecting the controller to a first interfacing portion of the isolator module 300a, 300b. In the first example provided above, a first cable 528 electrically connects the controller 518 of the surgeon's console 502 to the first interfacing portion of the isolator module 300a. In the second example provided above, a first cable 530 connects the controller 518 of the surgeon's console 502 to the first interfacing portion of the isolator module 300b. The first cable is configured to transfer power between the controller 518 and the first interfacing portion of the isolator module. The first cable is also configured to transfer data in both directions between the robot arm 506 and the first interfacing portion. The function and features of the first cable of the surgical robotic system have been described in further detail above.

The system further comprises a second cable 516, 532 connecting the robot arm 506 to a second interfacing portion of the isolator module 500a, 500b. In the first example provided above, a second cable 532 electrically connects the base 508 of the surgical robot 504a to the second interfacing portion of the isolator module 300a. In the second example provided above, a second cable 516 connects the surgical instrument 514 to the second interfacing portion of the isolator module 300b. The second cable 516, 532 is configured to transfer power between the robot arm 506 and the second interfacing portion of the isolator module 300a, 300b. The second cable 516, 532 is also configured to transfer data in both directions between the robot arm 506 and the second interfacing portion. The function and features of the second cable of the surgical robotic system have been described in further detail above.

The isolator module 300a, 300b is configured to perform in the same way as modules 300 and 400 illustrated in FIGS. 3 and 4 respectively. That is, the isolator module 300a, 300b is configured to transfer data and power between its first interfacing portion and its second interfacing portion whilst providing electrical isolation between the first interfacing portion and the second interfacing portion. More specifically, the isolator module 300a, 300b is configured to transfer data in both directions between its first interfacing portion and its second interfacing portion, and to transfer power in at least one direction from its first interfacing portion to its second interfacing portion, whilst providing electrical isolation between these two portions.

In an example, where the isolator module 300a is located at the surgeon's console, it may form part of an ethernet switchboard. The ethernet switchboard may be an isolated ethernet switchboard. The ethernet switchboard is located at the surgeon's console and comprises an ethernet switch, allowing it to perform similarly to a router. That is, data signals can be sent from the ethernet port of the ethernet switchboard to a number of different components within the surgeon's console, where each different component comprising its own ethernet port and its own IP address defining the data that it can and cannot receive. By combining the isolator module 300a with the ethernet switchboard of the surgeon's console, that the number of power distribution units in the surgeon's console can be reduced. This is because the power supply unit supplying power to the isolator module can be the same unit as the one that is supplying power to the ethernet switchboard. By reducing the number of power distribution units required, the hardware space available for constructing the other components and modules for the surgeon's console 502 can be increased.

In an alternative or a further exemplary arrangement to the arrangement illustrated in FIGS. 5 and 6, the surgical robotic system may comprise more than one isolator module that is configured to provide the same power and data links between the surgeon's console and one of the robot arms in that system. For example, a first isolator module may be located within a first surgical robot 504a, and a second isolator module may be located within the surgeon's console 102. The use of two isolator modules, instead of one, within the same data and power link further increases the isolation, amplification, and transfer speed capabilities of the isolator mechanism.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description, it will be evident to a person skilled in the art that various modifications may be made within the scope of the invention.

1. An isolator module for use in a surgical robotic system which comprises a robot arm and a controller, the isolator module being configured to provide an interface between the robot arm and the controller of the surgical robotic system, the isolator module comprising:
a first interfacing portion configured to: (i) transfer power from a first cable that is connected to the controller, and (ii) transfer data to and from the first cable;
a second interfacing portion configured to: (i) transfer power to a second cable that is connected to the robot arm, and (ii) transfer data to and from the second cable;
a third cable connecting the first interfacing portion to the second interfacing portion, the third cable being configured to transfer data in both directions between the first interfacing portion and the second interfacing portion whilst providing electrical isolation between the first interfacing portion and the second interfacing portion; and
an isolated power converter configured to transfer power from the first interfacing portion to the second interfacing portion whilst providing electrical isolation between the first interfacing portion and the second interfacing portion.

2. The isolator module of claim 1, wherein the first interfacing portion comprises a first control unit configured to:
transfer power received from the first cable to the isolated power converter; and
transfer data between the first cable and the third cable.

3. The isolator module of claim 1, wherein the second interfacing portion comprises a second control unit configured to:
transfer power received from the isolated power converter to the second cable; and
transfer data between the third cable and the second cable.

4. The isolator module of claim 1, wherein data is transmitted through the first and second cables according to a CoaXPress (CXP) data protocol.

5. (canceled)

6. The isolator module of claim 1, wherein the third cable is a fibre-optic cable that is configured to transfer data between the first interfacing portion and the second interfacing portion using light.

7. (canceled)

8. The isolator module of claim 1, wherein the data transferred from the robot arm to the controller comprises a stream of sensor data.

9. The isolator module of claim 8, wherein the first interfacing portion further comprises a signal conditioning module that is configured to receive a data stream from the third cable, extract an embedded clock from that data stream and retransmit a fresh version of the data stream to the first cable using a clean clock.

10. The isolator module of claim 9, wherein the first signal conditioning module is further configured to monitor the quality of the received data stream and transmit data indicating the quality of the received data stream.

11. The isolator module of claim 8, wherein the second interfacing portion further comprises a second signal conditioning module that is configured to receive a data stream from the second cable, extract an embedded clock from that data stream and retransmit a fresh version of the data stream to the third cable using a clean clock.

12. (canceled)

13. The isolator module of claim 1, wherein the isolated power converter is a DC-DC power converter.

14. The isolator module of claim 1, wherein each of the first and second interfacing portions comprises a small form-factor pluggable (SFP) interface module.

15. The isolator module of claim 1, wherein the module forms part of an isolated ethernet switchboard.

16. The isolator module of claim 10, further comprising a microcontroller connected to the first signal conditioning module and configured to receive the results of analysis performed by the first signal conditioning module and to use those analysis results to provide diagnostics information indicating the quality of data being transferred from the robot arm to the controller.

17. (canceled)

18. The isolator module of claim 16, wherein the diagnostics information provided by the microcontroller comprises one or more of the following indications:
an overcurrent and undercurrent of each of the first and second interfacing portions;
whether or not a first transceiver module and a second transceiver module are present on the isolator module, each of the first and second transceiver modules being configured to transmit and receive data signals to and from the third cable;
the status of the first and second transceiver modules;
whether the first signal conditioning module has been unlocked for use by the isolator module;
whether a second signal conditioning module has been unlocked for use by the isolator module;
whether there is a fault in the surgical robotic system;
the quality of the communication and the signal integrity of the first and second cables;
the temperature recorded from first and second temperature sensors located on the first and second interfacing portions, respectively; and
the current and voltage that are being provided to the surgical instrument, which thereby provides an indication of quality of the electrical connection to the surgical instrument.

19. The isolator module of claim 16, further comprising one or more indicators connected to the microcontroller, the one or more indicators being configured to indicate whether there is a fault in the surgical robotic system.

20. (canceled)

21. The isolator module of claim 1, wherein the robot arm comprises a surgical instrument with a camera.

22. (canceled)

23. The isolator module of claim 1, wherein the data transferred from the second interfacing portion to the first interfacing portion via the third cable includes one or more of diagnostics data, position data and sensor data.

24. (canceled)

25. (canceled)

26. The isolator module of claim 1, wherein the module is located in the surgical robotic system within a unit that comprises the robot arm.

27. The isolator module of claim 1, wherein the module is located in the surgical robotic system within a surgeon's console that comprises the controller.

28. A surgical robotic system comprising:
a controller;
a robot arm that is controlled by the controller;
an isolator module configured to provide an interface between the controller and the robot arm;
a first cable connecting the controller to a first interfacing portion of the isolator module, the first cable being configured to: (i) transfer power between the controller and the first interfacing portion, and (ii) transfer data between the controller and the first interfacing portion;
a second cable connecting the robot arm to a second interfacing portion of the isolator module, the second cable being configured to: (i) transfer power between the robot arm and the second interfacing portion, and (ii) transfer data between the robot arm and the second interfacing portion;
wherein the isolator module is configured to transfer data and power between the first interfacing portion and the second interfacing portion whilst providing electrical isolation between the first interfacing portion and the second interfacing portion.

29. (canceled)

30. (canceled)
